(19) United States
(12) Patent Application Publication
(10) Pub. No.: US 2010/0317886 A1
(43) Pub. Date: Dec. 16, 2010

(54) SYNTHESIS OF ISOTOPICALLY-LABELED FUNCTIONALIZED DIENES

(76) Inventor: Rodolfo A. Martinez, Santa Fe, NM (US)

Correspondence Address: Ortiz and Lopez, PLLC;Registered Patent Attorneys, P.O. Box 4484, Albuquerque, NM, 87196-4484, US

(21) Appl. No.: 12/813,797

(22) Filed: Jun. 11, 2010

Related U.S. Application Data
(60) Provisional application No. 61/186,334, filed on Jun. 11, 2009.

(51) Int. Cl. C07C 381/00; C07C 317/04; C07C 69/67

(52) U.S. Cl. 560/186; 568/75; 568/32

(57) ABSTRACT

All labeled carbons are derived ultimately from CO, as carbon-13 is separated from its lighter isotope by cyrogenic distillation of carbon monoxide (CO). Creation of stereospecific and site-specific compounds used for starting materials will address growing demands for labeled compounds, including isotopically-labeled functionalized dienes. Functionalized diene compounds can be used as precursors for the production of isotopically labeled pharmaceuticals, biomolecules, and natural products.

### CROSS REFERENCE TO RELATED APPLICATION

This patent application claims the priority and benefit of U.S. Provisional Patent Application 61/186,334 filed Jun. 11, 2009 entitled “Isotopically Labeled Precursors for Pharmaceutical Applications” that is herein incorporated by reference.

### TECHNICAL FIELD

The present invention relates to isotopically-labeled compounds and more particularly to compounds labeled with carbon-13 and hydrogen-2.

### BACKGROUND OF THE INVENTION

Stable, isotopically labeled, biologically active compounds are required for many phases of drug discovery and development including elucidation of biosynthetic pathways, pharmacokinetics, and drug metabolism. For many applications, site-specific ¹³C or combined ¹³C and ²H labeling are required. While a number of stable isotope-labeled compounds are available from companies such as Sigma-Aldrich Chemicals, a need remains for other labeled synthetic precursors.

All labeled carbons are derived ultimately from CO, as carbon-13 is separated from its lighter isotope by cyrogenic distillation of carbon monoxide (CO). The highly efficient conversion of CO to useful chemical precursors is perhaps the most unique aspect of stable isotope labeling technology. Any inefficiencies in the early synthetic steps add greatly to the overall expense of isotope labeling. Thus, considerable efforts have been directed to the development of methods for the preparation of useful synthetic precursors or synthons. This effort has given rise to efficient large-scale methods for the synthesis of methane, methanol, methyl iodide, sodium formate, potassium cyanide, and carbon dioxide. These methods are the foundation of all labeling chemistry.

As spectroscopic instrumentation and techniques continue to improve, there is a drive to study ever more complicated bio-systems that demand more complex labeling patterns in biomolecules. Additionally, the use of stable isotopes has proven to be a promising analytical tool that has driven a need for isotopically labeled compounds. In the past, the simple introduction of a site-specifically labeled atom without stereospecificity was the major thrust for stable isotope labeling. The first generation of labeled synthons served this effort well. Increasingly though, in today's labeling climate, stereospecificity is required, along with prior site-specific labeling efforts. Stereospecificity includes the ability to both stereospecific label chiral compounds, as well as differentiate between prochiral centers with deuterium or carbon. Creation of stereospecific and site-specific compounds used for starting materials will address those growing demands.

Such in-demand compounds include isotopically-labeled functionalized dienes. Dienes have a great synthetic importance and are used extensively to produce a wide variety of compounds. There are over 25,000 literature references illustrating the use of dienes for the construction of complicated chemical compounds including the following, which are incorporated herein by reference:
1. Danishefsky, S.; Harayama, T.; Singh, R. K., Use of CE≦-phenylsulfinyl-CE±,CE≦-unsaturated carbonyl dienophiles in Diels-Alder reactions. J. Am. Chem. Soc. 1979, 101 (23), 7008-12.
2. Danishefsky, S.; Hirama, M., Total synthesis of disodium prephenate. J. Am. Chem. Soc. 1977, 99 (23), 7740-1.
3. Danishefsky, S.; Hirama, M.; Gombatz, K.; Harayama, T.; Berman, E.; Schuda, P. F., Total synthesis of dl-pentalenolactone. J. Am. Chem. Soc. 1979, 101 (23), 7020-31.
4. Danishefsky, S.; Kerwin, J. F., Jr., On the Lewis acid catalyzed cyclocondensation of silyloxydienes with CE±,CE≦-unsaturated aldehydes. J. Org. Chem. 1982, 47 (16), 3183-4.
5. Danishefsky, S.; Kerwin, J. F., Jr.; Kobayashi, S., Lewis acid catalyzed cyclocondensations of functionalized dienes with aldehydes. J. Am. Chem. Soc. 1982, 104 (1), 358-60.
6. Danishefsky, S.; Kitahara, T.; Schuda, P. F., Preparation and Diels-Alder reaction of a highly nucleophilic diene: trans-1-methoxy-3-(trimethylsiloxy)-1,3-butadiene. (Silane, [(3-methoxy-1-methylene-2-propenyl)oxy]trimethyl-). Org. Synth. 1983, 61, 147-51.
7. Danishefsky, S.; Kitahara, T.; Yan, C. F.; Morris, J., Diels-Alder reactions of trans-1-methoxy-3-trimethylsilyloxy-1,3-butadiene. J. Am. Chem. Soc. 1979, 101 (23), 6996-7000.
8. Danishefsky, S.; Prisbylla, M. P.; Hiner, S., The use of trans-methyl CE≦-nitroacrylate in Diels-Alder reactions. J. Am. Chem. Soc. 1978, 100 (9), 2918-20.
9. Danishefsky, S.; Webb, R. R., II, Lewis acid catalyzed cyclocondensations of formaldehyde with activated dienes. A direct route to pyranosidal pentoses. J. Org. Chem. 1984, 49 (11), 1955-8.
10. Kerwin, J. F., Jr.; Danishefsky, S., On the Lewis acid catalyzed cyclocondensation of imines with a siloxydiene. Tetrahedron. Lett. 1982, 23 (37), 3739-42.
11. Danishefsky, S.; Hirama, M.; Gombatz, K.; Harayama, T.; Berman, E.; Schuda, P. Stereospecific total synthesis of dl-pentalenolactone. J. Am. Chem. Soc. 1978, 100 (20), 6536-8.
12. Danishefsky, S.; Kerwin, J. F., Jr., A simple synthesis of dl-chalcose. J. Org. Chem. 1982, 47 (8), 1597-8.
13. Danishefsky, S.; Kitahara, T.; McKee, R.; Schuda, P. F., Reactions of silyl enol ethers and lactone enolates with dimethyl(methylene)ammonium iodide. The bis-CE±-methylenation of pre-vernolepin and pre-vernomenin. J. Am. Chem. Soc. 1976, 98 (21), 6715-17.
14. Danishefsky, S.; Kitahara, T.; Schuda, P. F.; Etheredge, S. J., A remarkable epoxide opening. An expeditious synthesis of vernolepin and vernomenin. J. Am. Chem. Soc. 1976, 98 (10), 3028-30.
15. Danishefsky, S.; Kobayashi, S.; Kerwin, J. F., Jr., Cram rule selectivity in the Lewis acid catalyzed cyclocondensation of chiral aldehydes. A convenient route to chiral systems of biological interest. J. Org. Chem. 1982, 47 (10), 1981-3.
16. Danishefsky, S.; Morris, J.; Clizbe, L. A., The conversion of L-glutamate to L-tyrosine. Heterocycles 1981, 15 (2), 1205-7.
17. Danishefsky, S.; Morris, J.; Clizbe, L. A., Total synthesis of pretyrosine (arogenate). J. Am. Chem. Soc. 1981, 103 (6), 1602-4.
18. Danishefsky, S.; Schuda, P. F.; Kitahara, T.; Etheredge, S. J., The total synthesis of dl-vernolepin and dl-vernomenin. J. Am. Chem. Soc. 1977, 99 (18), 6066-75.
19. Danishefsky, S. J.; Balog, A.; Bertinato, P.; Su, D.-S.; Chou, T.-C.; Meng, D. F.; Kamenecka, T.; Sorensen, E. J. Synthesis of epothilones, intermediates and analogs for use in treatment of cancers with multidrug-resistant phenotype. 97-US22381 9901124, 19971203, 1999.
20. Danishefsky, S. J.; Bornmann, W. G.; Queneau, Y.; Magee, T. V.; Krol, W. J.; Masters, J. J.; Jung, D. K. Total synthesis of taxol and its analogs. 94-US12661 9512567, 19941102, 1995.
21. Danishefsky, S. J.; Pearson, W. H.; Harvey, D. F., Diastereofacial control in the Lewis acid catalyzed cyclocondensation reaction of aldehydes with activated dienes: a synthesis of the Mus musculus pheromone. J. Am. Chem. Soc. 1984, 106 (8), 2455-6.
22. Queneau, Y.; Krol, W. J.; Bornmann, W. G.; Danishefsky, S. J., Nozaki-Kishi reaction of crotonates as a source of complex dienophiles. Application to the B-seco taxane series. Bull. Soc. Chim. Fr. 1993, 130 (3), 358-70.
23. Yoshino, T.; Ng, F.; Danishefsky, S. J., A Total Synthesis of Xestodecalactone A and Proof of Its Absolute Stereochemistry: Interesting Observations on Dienophilic Control with 1,3-Disubstituted Nonequivalent Allenes. J. Am. Chem. Soc. 2006, 128 (43), 14185-14191.

In order to meet the urgent and growing demand for labeled pharmaceuticals, biomolecules, and natural products, high purity, isotopically-labeled diene compounds are needed.

### BRIEF SUMMARY

The following summary is provided to facilitate an understanding of some of the innovative features unique to the embodiments and is not intended to be a full description. A full appreciation of the various aspects of the embodiments can be gained by taking the entire specification, claims, drawings, and abstract as a whole.

It is therefore an aspect of the embodiments to provide isotopically-labeled diene compounds. The diene compounds can be used as precursors for the production of isotopically labeled pharmaceuticals, biomolecules, and natural products.

### BRIEF DESCRIPTION OF THE DRAWINGS

The accompanying figures, in which like reference numerals refer to identical or functionally similar elements throughout the separate views and are incorporated in and form a part of the specification, further illustrate aspects of the embodiments and, together with the background, brief summary, and detailed description, serve to explain the principles of the embodiments.

FIG. 1 illustrates an isotopically-labeled functionalized diene in accordance with the disclosed embodiments;

FIG. 2A illustrates a high yield synthetic route for the production of ethyl-3-ethoxy-2-[¹³C₃]propenoate in accordance with the disclosed embodiments;

FIG. 2B illustrates isotopically-labeled 3,3-diethoxy[U—¹³C]propionic acid in accordance with the disclosed embodiments;

FIG. 2C illustrates isotopically-labeled 3-ethoxy[U—¹³C]-acrylic acid in accordance with the disclosed embodiments;

FIG. 3 illustrates obtaining isotopically labeled 4,4-diethoxy-2-[1-¹³C]butenoate and 4-ethoxy-2-[1-¹³C]butanone in accordance with the disclosed embodiments;

FIG. 4 illustrates obtaining uniformly-labeled 4,4-diethoxy-2-[U—¹³C₄]butanone using ethyl 3,3-diethoxy-2-[U—¹³C₃]butenoate and [¹³C]methylphenylsulfoxide in accordance with the disclosed embodiments;

FIG. 5 illustrates obtaining 4-ethoxy-3-[U-13C4]butene-2-one in accordance with the disclosed embodiments;

FIG. 6 illustrates obtaining trans-4-hydroxy-2-buteneoic acid in accordance with the disclosed embodiments;

FIG. 7 illustrates obtaining cis and trans 4-hydroxy-2-butenoic acid in accordance with the disclosed embodiments;

FIG. 8 illustrates obtaining (E,Z) 1-methoxy-3-t-butyldimethylsiloxy-4-(phenylsulfinyl)[4-¹³C]1,3-butadiene in accordance with the disclosed embodiments;

FIG. 9 illustrates a general formula for certain labeled compounds in accordance with the disclosed embodiments;

FIG. 10 illustrates another general formula for certain labeled compounds in accordance with the disclosed embodiments;

FIG. 11 illustrates another general formula for certain labeled compounds in accordance with the disclosed embodiments; and

FIG. 12 illustrates yet another general formula for certain labeled compounds in accordance with the disclosed embodiments.

### DETAILED DESCRIPTION

The following description contains a series of examples wherein previously known unlabeled compounds are processed to yield highly pure labeled compounds that are not previously known.

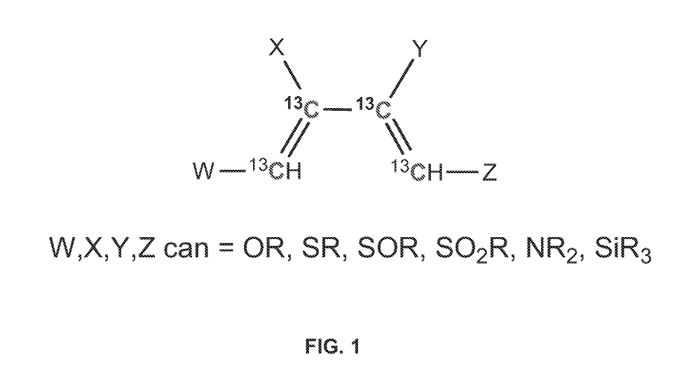

FIG. 1 illustrates an isotopically-labeled conjugated diene, wherein W, X, Y, and Z can be selected from the group consisting of OR, SR, SOR, SO₂R, NR₂, and SiR₃. R can be selected from the group consisting of H, alkyl, aryl, phenyl, or benzyl. Conjugated dienes undergo a cycloaddition reaction with multiple bonds to form unsaturated six-membered rings. This is a 1,4-addition of a conjugated diene and a dienophile. The unlabeled title compounds have been synthesized from alkyl-3-alkoxy-2-butenoates. A new route for the synthesis of isotopically labeled alkyl 3-alkoxy-2-butenoates was developed.

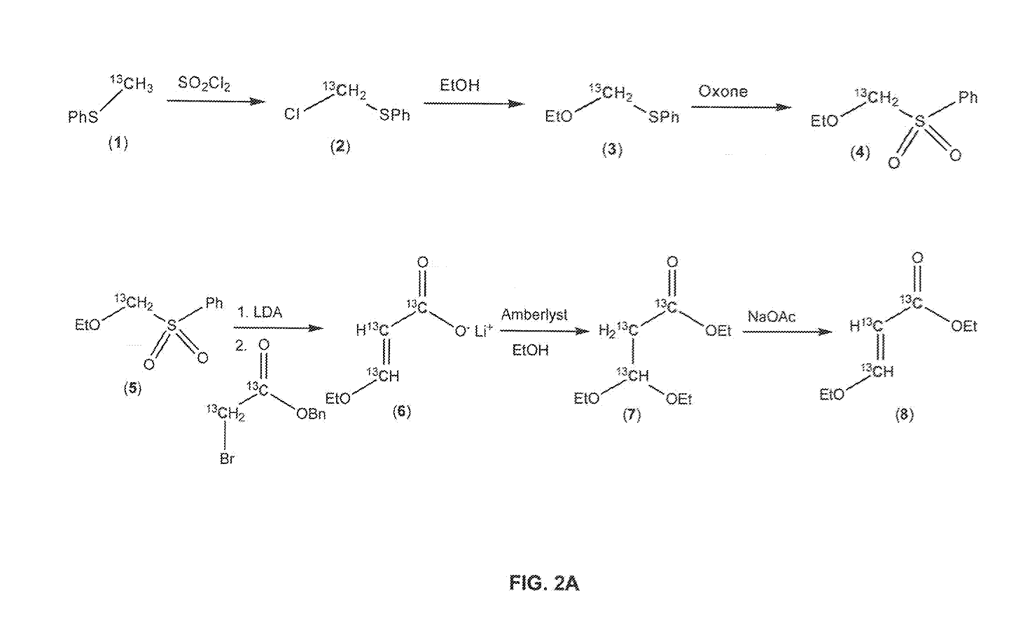

FIG. 2A illustrates a high-yield synthetic route for the production of ethyl-3-ethoxy-2-[¹³C₃]propenoate (7) in accordance with the disclosed embodiments, as follows:

### Synthesis of ethoxy[¹³C]methylphenyl sulfide (3)

Chloro[¹³C]methylphenyl sulfide (12.0 g, 0.075 mol) and ethyl alcohol (120 mL, 100%) were placed in a 250 mL round bottom flask equipped with a magnetic stir bar and a rubber septum. This mixture was sonicated at 40° C. for 6 hours and then allowed to stir overnight at room temperature without sonication. After this period, ¹³CNMR analysis showed the complete disappearance of the starting material at 51 ppm and the quantitative formation of the desired product at 74 ppm. The reaction mixture was then transferred to a separatory funnel containing dichloromethane (120 mL) and DI water (100 mL). The organic layer was separated, dried over anhydrous sodium sulfate, filtered, and then concentrated in vacuo to afford 12.46 g (98.2%) of the titled compound as a pale yellow fluid. The crude product was sufficiently pure and was used in the next reaction without further purification.

The spectra data are as follows:

¹HNMR (300 MHz in CDCl₃ with 0.03% TMS) δ: 7.48-7.18 (5H, m), δ: 5.23, 4.70 (2H, d J 158.1) δ: 3.68, 3.67, 3.66, 3.65, 3.63, 3.62, 3.61, 3.60 (2H, qd J 6.98, 3.67) δ: 1.23, 1.19, 1.18 (3H, t J 6.99).

¹³CNMR (75 MHz in CDCl₃) δ: 136.398, 130.23, 129.00, 126.71, 74.70, 63.93, and 15.80; Mass spectra m/e. 169 (M+.), 124, 109, and 60.

### Synthesis of ethoxy[¹³C]methylphenyl sulfones (4)

An oxone solution (prepared by dissolving 163.69 g of oxone in 720 ml deionized water) was added to an ice-cooled solution of ethoxy[1-¹³C]-methylphenyl sulfide (16.0 g, 0.095 mol) in ethyl acetate-ethanol (1:1, 150 mL). This reaction mixture was allowed to stir at 0° C. for 30 minutes and ¹³CNMR in CDCl₃ at that point showed a quantitative formation of the desired product peak at 86 ppm. The reaction mixture was poured into a 2-L separatory funnel containing dichloromethane (300 mL) and deionized water (350 mL). The organic layer was separated and washed with deionized water (3×150 mL). The combined organic layers were dried over anhydrous sodium sulfate, filtered, and then concentrated in vacuo to afford 18.4 g, (96.7%) of the titled compound as pale yellow fluid pure enough for the next reaction.

The spectra data are as follows:

¹HNMR (300 MHz in CDCl₃ with 0.03% TMS)/δ: 7.95-7.55 (m 5H), δ: 4.81, 4.30 (d J 154.43 ¹³CH₂); δ: 3.93, 3.91, 3.89, 3.90, 3.89, 3.87, 3.86, 3.85, 3.84 (qd J 6.99, 3.31 —OCH₂) δ: 1.21, 1.19, 1.16 (t J 6.99).

¹³CNMR (75 MHz in CDCl₃) δ: 134.99, 134.20, 129.36, 128.97 δ: 86.43 (s, ¹³CH₂) δ: 69.53, (—OCH₂), and 15.20 (CH₃).

### Synthesis of ethyl-3,3-diethoxy[U—¹³C]propionate (7)

Ethoxy[1¹³C]-methylphenyl sulfone (15.0 g, 0.075 mol) and dry tetrahydrofuran (THF) (150 mL) were placed in a 250 mL round bottom flask equipped with a magnetic stir bar and a rubber septum fitted to nitrogen inlet. The resultant solution was purged under a constant flow of nitrogen after which it was submerged in an ethanol/dry ice bath bringing it to a temperature of −78° C. The solution was then equilibrated at that temperature by allowing it to stir for a period of 15 minutes. Lithium diisopropyl amide (LDA) (109.4 mL, 0.164 mol.) was added slowly via a syringe to the mixture. The reaction mixture was stirred for 45 minutes to ensure complete anion formation. At that point, a solution of [U—¹³C₂]bromoacetic acetic acid (11.56 g, 0.0825 mol in THF (15 mL)) was added slowly to the reaction mixture. This mixture was allowed to stir for an additional hour. To this reaction mixture was added another portion of LDA (54.7 mL, 0.0835 mol.). ¹³CNMR of an aliquot taken in D₂O, showed the quantitative formation of ethoxy[U—¹³C₃]acrylate. After stirring for an additional hour, the mixture was partitioned between dichloromethane (125 mL) and deionized water (200 mL). The aqueous layer was separated and poured into a separatory funnel containing dichloromethane (125 mL). This mixture was then acidified with 1N HCl to a pH 2 and the organic layer was separated, dried over anhydrous sodium sulfate, filtered, and then concentrated under reduced pressure which afforded 13.25 g of a crude mixture of trans-ethoxy[U—¹³C₃]-propenoic acid and benzene sulfinic acid. This crude mixture was immediately dissolved in absolute ethanol (200 mL) and after about 5 mins of stirring, (9.0 g) was added. The entire mixture was then heated to reflux for 4 hrs. After this period, ¹³CNMR of an aliquot taken in CDCl₃ showed the complete formation of the desired product. The heating was discontinued and the flask was allowed to cool to room temperature. The residual Amberlyst® ion exchange resin was filtered off using a frit funnel packed with celite, then the celite cake was rinsed with dichloromethane (2×25 mL). The resultant solution was poured into a separatory funnel containing hexane (150 mL) and deionized water (300 mL). The hexane layer was separated, filtered into a round bottom flask, and concentrated using a rotary evaporator set at 25° C., 75 torr, which gave 15.78 g as a mixture of ethyl phenylsulfinate and the titled compound as pale yellow oil. The entire crude was chromatographed by DCC to afford 8.89 g, 61.35% of the title compound as a pale yellow liquid which was used in subsequent reactions without further purification.

The spectra data are as follows:

¹HNMR (300 MHz in CDCl₃ with 0.03% TMS)/δ: 5.249, 5.244, 5.228, 5.225, 5.209, 5.205, 4.701, 4.698, 4.678, 4.661 (dtd, ¹³CH J 164.36, 6.98, 1.46); δ: 4.197, 4.186, 4.173, 4.163 4.149, 4.138, 4.126, 4.115 (qd 2H J 7.36, 3.31 Hz); δ: 4.162-3.49 (two unresolved qd which appears as a multiplet 4H); δ: 2.903, 2.881, 2.861, 2.839, 2.469, 2.448, 2.426, 2.406 (ddd ¹³CH₂, J 130.16, 12.87, 6.62 Hz); δ: 1.288, 1.265, 1.241, 1.224, 1.200, 1.177 (two sets of triplets 9H J 6.97 Hz).

¹³CNMR (75 MHz in CDCl₃) δ: 170.583, 169.802, (d ¹³COOEt, J 58.86 Hz), δ: 100.149, 99.543 (d ¹³CH J 45.77 Hz); δ: 40.850, 40.229, 40.070, 39.464 (dd ¹³CH₂ 58.86, 45.77 Hz).

### Synthesis of 3,3-diethoxy[U—¹³C]propionic acid

A 50/50 mixture of ethyl-3,3-diethoxy[U—¹³C]propionate and benzene sulfinic acid ethyl ester (8.89 g) was treated with 1N NaOH (70 mL) in a 250 mL round bottom flask. The mixture was stirred at room temperature for an hour, after which it was poured into a separatory funnel containing dichloromethane (50 mL). This mixture was then acidified and extracted with dichloromethane at pH values of 6, 4, 2 and 1. The organic layers extracted at pH values of 4 and 6 were combined, dried over anhydrous sodium sulfate, filtered, and then concentrated in vacuo to afford 3.1 g, (81.36%) of the titled compound as pale yellow oil. This reaction was used as a purification technique for ethyl 3,3-diethoxy[U—¹³C]propionate. Isotopically-labeled 3,3-diethoxy[U—¹³C]propionic acid is illustrated in FIG. 2B.

The spectra data are as follows:

¹HNMR (300 MHz in CDCl₃ with 0.03% TMS)/δ: 10.67 (s 1H), δ: 5.24, 5.22, 5.20, 4.69, 4.67, 4.66 (dtd J 165.82, 5.88, 1.47 1H), δ: 3.78-3.51 (unresolved multiplet), δ: 2.96, 2.94, 2.92, 2.90, 2.53, 2.51, 2.48, 2.47 (ddd J 129.79, 12.87, 5.88 2H), δ: 1.23, 1.21, 1.18 (t J 7.35 Hz).

¹³CNMR (75 MHz in CDCl₃) δ: 175.79, 175.04 (d J 56.68) δ: 99.51, 98.90 (d J 45.78), δ: 62.00, δ: 40.30, 39.69, 39.56, 38.94 (dd J 56.68, 45.79) δ: 15.10, 15.05 (d J 3.27).

### Esterification of 3,3-diethoxy-[U—¹³C]propionic acid to ethyl-3,3-diethoxy[U—¹³C]propionate

3,3-Diethoxy[U—¹³C]propionic acid (1.5 g, 9.0 mmol), Amberlyst® ion exchange resin (3.5 g) and absolute ethanol (15 mL) were placed in a 100 mL round bottom flask equipped with a reflux condenser and a magnetic stir bar. This mixture was heated to reflux with constant stirring for 4 hours. ¹³CNMR of an aliquot taken in CDCl₃ indicated the complete formation of the desired product. The heating was discontinued and the flask was allowed to cool to room temperature. The residual Amberlyst® ion exchange resin was filtered off using a frit funnel packed with celite, and then the celite cake was rinsed with dichloromethane. The resultant solution was poured into a separatory funnel containing hexane (35 mL) and DI water (40 mL). The hexane layer was separated, dried over anhydrous sodium sulfate, filtered, and then concentrated using a rotary evaporator set at 25° C., 75 torr which gave 1.52 g, 87.34% of the titled compound as yellow liquid. The crude obtained from this reaction was used in subsequent reactions without further purification.

The spectra data are as follows:

¹HNMR (300 MHz in CDCl₃ with 0.03% TMS)/δ: 5.25, 5.24, 5.23, 5.22, 5.21, 5.20, 4.70, 4.69, 4.67, 4.66 (dtd, ¹³CH J 164.36, 6.98, 1.46); δ: 4.19, 4.18, 4.17, 4.16 4.14, 4.14, 4.13, 4.12 (qd 2H J 7.36, 3.31). δ: 4.16-3.49 (two unresolved qd which appears as a multiplet 4H); δ: 2.90, 2.88, 2.86, 2.84, 2.47, 2.45, 2.43, 2.41 (ddd ¹³CH₂, J 130.16, 12.87, 6.62); δ: 1.29, 1.27, 1.24, 1.22, 1.20, 1.18 (two sets of triplets 9H J 6.97).

¹³CNMR (75 MHz in CDCl₃) δ: 170.58, 169.80, (d ¹³COOEt, J=58.86 Hz), δ: 100.15, 99.54 (d ¹³CH J 45.77 Hz), δ: 40.85, 40.23, 40.07, 39.46 (dd ¹³CH₂ 58.86, 45.77 Hz).

### Synthesis of 3-ethoxy[U—¹³C]-acrylic acid

Diethoxy[U—¹³C]propionic acid (80 wt %, 1.17 g, 0.0057 mol) and dry tetrahydrofuran (10 mL) were placed in an oven dried 100 mL round bottom flask equipped with a magnetic stir bar and a rubber septum fitted to a nitrogen inlet. This mixture was then subjected to a constant flow of nitrogen and equilibrated at a temperature of −12° C. by submerging in an ethanol/ice bath. After about 10 minutes, a THF solution of lithium diisopropyl amide (10.5 mL, 15.75 mmol) was added. ¹³CNMR analysis of an aliquot taken in D₂O showed the complete formation of the desired product. The reaction mixture was then transferred into a 250 mL separatory funnel containing dichloromethane (30 mL) and DI water (10 mL). This mixture was then acidified with 1N HCl to a pH of 2 and the organic layer was separated, dried over anhydrous sodium sulfate, filtered, and concentrated in vacuo to afford 0.58 g, 75% of [1, 2, 3, ¹³C₃]-3-ethoxy acrylic acid. Isotopically-labeled 3-ethoxy[U—¹³C]-acrylic acid is illustrated in FIG. 2C.

The spectra data are as follows:

¹HNMR (300 MHz in CDCl₃ with 0.03% TMS) δ: 10.42 (s OH), δ: 8.02, 8.00, 7.99, 7.98, 7.97, 7.96, 7.95, 7.93, 7.92, 7.40, 7.39, 7.38, 7.37, 7.36, 7.35, 7.34, (dddd J 183.1, 12.13, 6.25, 3.31, 1H), δ: 5.47, 5.46, 5.43, 5.42, 4.93, 4.91, 4.88, 4.87, (ddd J 163.25, 12.5, 3.68 1H), δ: 3.99, 3.98, 3.96, 3.95, 3.94, 3.93, 3.92, 3.91 (qd J 2.58, 6.99 2H), δ: 1.38, 1.35, 1.33 (t J 6.98 3H).

¹³CNMR (75 MHz in CDCl₃ with 0.03% TMS) δ: 174.30, 174.24, 174.28, 173.21 (dd J 77.39, 77.38), δ: 165.00, 164.922, 163.96, 163.89 (dd J 78.47, 77.38), δ: 96.64, 95.60, 94.57 (dd J 78.47, 77.38) δ: 67.69, 67.25, 67.06, 66.48 (dd J 47.96, 57.77) and δ: 14.34.

### Synthesis of alkyl 3-ethoxy[U—¹³C]propenoate (8)

3-Ethoxy-2-[3-¹³C]propenoate lithium carboxylate (9.19 g, 74.6 mmol, 1.0 equivalent) was in an aqueous/THF solution that was rotovaped down at 35° C. in a two-hundred and fifty milliliter Morton flask. The resulting brown solid was then dissolved in DMF (90 mL) before methyl iodide (5.17 mL, 82.1 mmol, 1.1 eq) was added slowly while stirring vigorously at room temperature. After 115 hours and a total of 4.4 eq of methyl iodide added, the reaction was found to be complete by ¹³CNMR by taking an aliquot from the reaction mixture, dissolving it into CH₂Cl₂ and H₂O, and separating the two layers. The disappearance (to within <2% remaining), of 3-ethoxy-2-[3-¹³C]propenoate lithium carboxylate (δ=157 ppm) in the aqueous layer and subsequent appearance of the desired 3-ethoxy-methyl-2-[3-¹³C]propenoate in the organic layer (δ=163 ppm) was monitored. The reaction mixture was worked up by filtering off solids. The filtrate was then vacuum distilled to give a clear liquid (6.26 g, 64%). The crude product was used without further purification.

The spectra data are as follows:

¹HNMR (CDCl₃, 300 MHz): δ=1.6-1.9 (t, 3H), 4.0-4.2 (s, 3H), 4.3-4.5 (m, 2H), 5.5-5.7 (m, 1H), 7.6-7.8 (d, 0.5H), 8.2-8.5 (d, 0.5H).

¹³CNMR (CDCl₃, 75 MHz): δ=15.5, 15.8 (d, CH₃), 51.07 (CH₃), 60.3, 60.5 (d, CH₂), 95.5, 96.6 (d, CH), 162.5 (¹³CH), 168.3 (C═O).

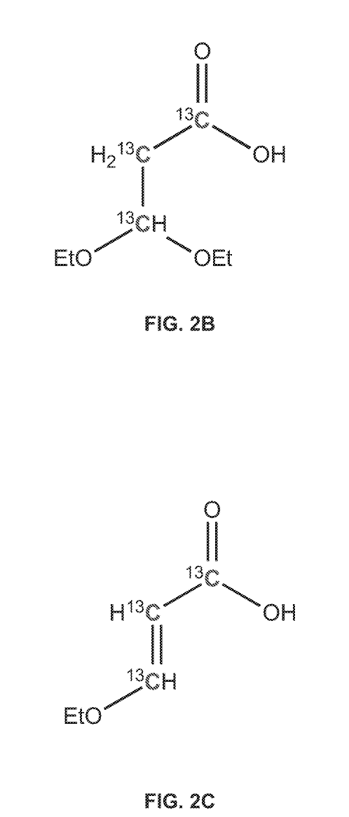

FIG. 3 illustrates obtaining isotopically labeled 4,4-diethoxy-2-[1-¹³C]butenoate (10) and 4-ethoxy-2-[1-¹³C]butanone (11) in accordance with the disclosed embodiments. FIG. 3 shows the versatility of the synthetic sequence for the production of any isotopomeric combination by one simple route for the production of isotopically labeled 4,4-diethoxy-2-[1-¹³C]butenoate (10) and 4-ethoxy-2-[1-¹³C]butanone (11). Because the compounds are assembled, in most cases, by one carbon additions starting from [¹³C]methylphenylsulfide, as one carbon source, and 1-[¹³C], 2-[¹³C], or 1,2-[¹³C₂]-bromoacetic acid as the other carbon source, all isotopic combinations are easily produced as follows:

### Synthesis of 4, 4 diethoxy-1-(phenylsulfinyl) [1-¹³C]butan-2-one (10)

[1-¹³C]-Methyl phenyl sulfoxide (3.0 g, 0.021 mol) and anhydrous tetrahydrofuran (20 mL) were placed in a 250 mL oven dried round bottom flask equipped with a magnetic stir bar and a rubber septum fitted to a nitrogen inlet. This mixture was subjected under a constant flow of nitrogen after which it was equilibrated at −78° C. for 10 minutes in an ethanol (absolute)/dry ice bath. Lithium diisopropylamide (18.4 mL, 0.027 mol 1.3 eq) was added slowly to the mixture. After about 45 minutes of stirring, ethyl-3,3-diethoxy propionate (9) (90 wt %, 4.25 g, 4.38 mL, and 0.022 mots) was added neat to the reaction mixture. Initial NMR showed the formation of an intense peak at 69 ppm, indicative of the product and some starting material at 44 ppm (a ratio of 85% to 15% product starting material respectively). The entire mixture was allowed to stir for a period of 4.0 hours after which it was partitioned between dichloromethane (75 mL) and deionized water (30 mL). The aqueous layer was separated and transferred into a separatory funnel containing dichloromethane (50 mL). This mixture was acidified to a pH of 2, and then the organic layer was separated and thoroughly washed with DI water (2×100 mL). The combined organic layers were dried over anhydrous sodium sulfate, then filtered and concentrated using a rotary evaporator to afford 5.1 g, 85.3% of the titled compound as a red liquid that was used without further purification.

The spectra data are as follows:

¹HNMR δ: 7.68-7.52 (5H, m), δ: 4.843, 4.824, 4.87 (1H, t J 5.51), δ: 4.226, 4.178, 4.154, 3.75 (1H, dd J 140.83, 14.36), δ: 4.216, 4.167, 4.106, 3.71 (1H dd J 140.82, 14.71) δ: 3.689, 3.680, 3.662, 3.655, 3.648, 3.639, 3.632, 3.625, 3.615, 3.607, 3.601, 3.584, 3.578, 3.552, 3.534, 3.529, 3.522, 3.511, 3.506, 3.498, 3.487, 3.480, 3.474, 3.64, 3.457. (4H, two quartets that appear as a multiplet), δ: 2.856, 2.824, 2.793, 2.774, 2.724, (2H ddd J 15.07, 9.92, 4.05), δ: 1.199, 1.182, 1.176, 1.158, 1.154, 1.135 (6H, t J 5.70).

¹³CNMR (75 MHz in CDCl₃) δ: 199.30, 198.78 (d J 41.41 C═O), 143.16, 131.56, 129.44, 124.12 (aromatic carbons) δ: 99.355 (CH) δ: 69.439 (¹³CH₂), δ: 62.49, 62.47 (—OCH₂) 49.25, 49.09 (—CH₂) 15.18.

### Synthesis of 4,4-diethoxy-2[1¹³C]-butanone (12)

A solution of 4,4-diethoxy-1-(phenylsulfinyl)[1-¹³C]butan-2-one (0.5 g 1.75 mmol) and ethanol (5 mL absolute) was stirred at room temperature under a constant flow of nitrogen and a scoop of wet Raney nickel 2800 was added. The reaction mixture immediately changed from a yellow to an orange color. TLC analysis (80% EtOAc/20% Hexane) at that point showed the presence of some starting material. After about 30 minutes of reaction time, another scoop of Raney nickel was added. The reaction mixture changed from an orange to milky appearance and TLC analysis showed the complete disappearance of starting material. The Raney nickel was filtered using a frit funnel packed with celite, and the celite cake was rinsed continuously with ethanol. The filtrate was partitioned between dichloromethane (20 mL) and DI water (10 mL) and the organic layer was separated, dried over anhydrous sodium sulfate, filtered into a round bottom, and then concentrated under reduced pressure to afford 0.25 g, 89.2% of a pale yellow fluid. (Rf=0.48, 80% Hex/20% EtOAc).

The spectra data are as follows:

¹HNMR (300 MHz in CDCl₃ with 0.03% TMS) δ: 4.92, 4.90, 4.88 (t J 5.53 1H), δ: 3.69, 3.68, 3.67, 3.66, 3.65, 3.64, 3.61, 3.58, 3.56, 3.55, 3.53, 3.51, 3.50, (m pseudo chirality effect, 4H), δ: 2.76, 2.74 (d J 5.88 2H), δ: 2.39, 1.97 (d, J 127.21 ¹³CH₃), δ: 1.22, 1.19, 1.17 (t, J 6.98, 3H).

¹³CNMR (75 MHz in CDCl₃) δ: 205.96, 205.43 (d J 40.33 C═O), δ: 99.73, (CH(OR)₂) δ: 62.14, δ: 48.30, 48.13 (d J 13.08 CH₂) 31.06 (s ¹³CH₃), and 15.12.

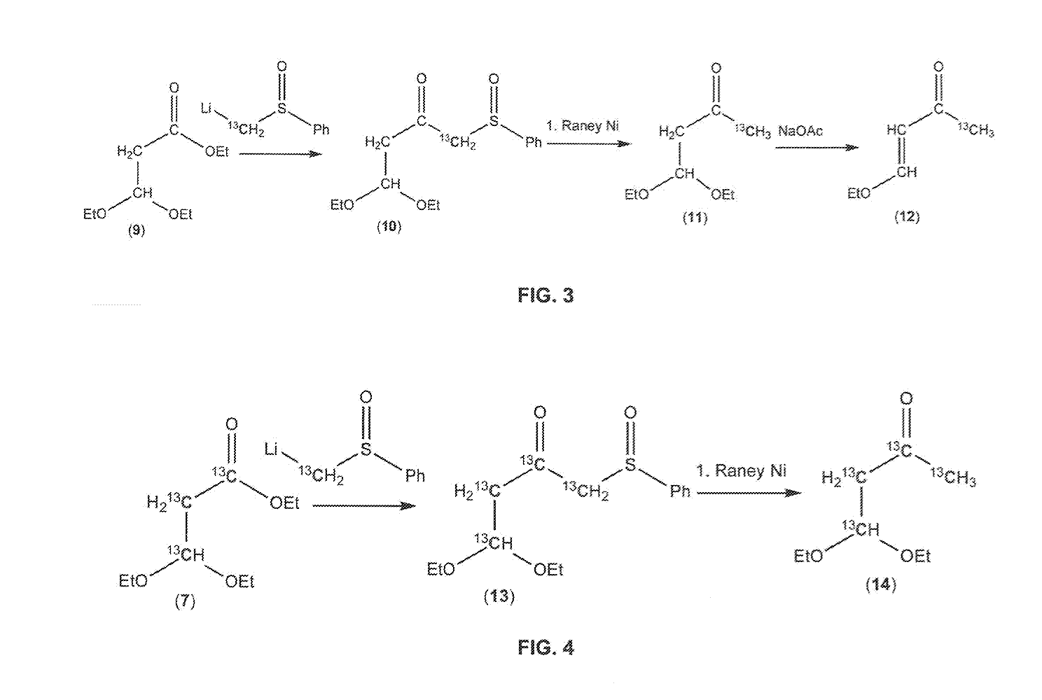

FIG. 4 illustrates obtaining uniformly-labeled 4,4-diethoxy-2-[U—¹³C₄]butanone (13) using ethyl 3,3-diethoxy-2-[U—¹³C₃]butenoate (7) and [¹³C]methylphenylsulfoxide in accordance with the disclosed embodiments. Uniform labeled 4,4-diethoxy-2-[U—¹³C₄]butanone (13) has been produced by starting with ethyl 3,3-diethoxy-2-[U—¹³C₃]butenoate (7) and [¹³C]methylphenylsulfoxide as shown in FIG. 4, as follows:

### General procedure for the synthesis of trans-4-alkoxy-3-[1-¹³C]butene-2-one (12, 15)

4,4-diethoxy[1-¹³C]butan-2-one and sodium acetate in toluene were heated under reflux and stirring for 14 hours. The product is produced in quantitative yield.

### Synthesis of 4, 4 diethoxy-1-(phenyl sulfinyl)[U—¹³C₄]butan-2-one (13)

The same procedure as above in the synthesis of 4,4-diethoxy-2[1-¹³C]-butanone (12) was repeated using [¹³C]-methylphenyl sulfoxide (0.93 g, 6.5 mmol), ethyl 3,3-diethoxy[U¹³C₃]propionate (1.4 g, 7.3 mmol) and LDA (6.5 mL, 9.75 mmol). This reaction afforded 1.59 g, 85% of the titled compound as a yellow fluid. This crude product was used in the next reaction without further purification.

The spectra data are as follows:

¹HNMR δ: 7.68-7.51 (m 5H), δ: 5.11-4.52 (dtd J 163.62, 5.51, 1.84 1H), δ: 4.23-3.75 (ddd J 140.46 13.98 4.42, 1H), δ: 3.74-3.42 (unresolved multiplets), δ: 3.08-2.50 (dddd J 128.32, 15.07, 9.19, 5.14, 2H), δ: 1.19-1.13 (t 3H).

¹³CNMR (75 MHz in CDCl₃) δ: 199.19, 198.96, 198.41, (dd J 40.33, 41.41 ¹³C═O), δ: 143.10, 131.51, 129.39, 124.09 (aromatic carbons); δ: 99.63, 99.02 (d 45.78, ¹³CH), δ: 69.43, 69.27, 68.91, 68.75 (dd, J 39.24, 39.24, PhSO¹³CH₃); 62.46 (OCH₂); δ: 49.79, 49.63, 49.24, 49.19, 49.08, 49.04, 48.48, (ddd 45.78, 41.42, 11.99 ¹³CH₂); δ: 15.15, 15.10 (d 3.27 CH₃).

### Synthesis of 4,4-diethoxy-2[1-¹³C]-butanone (14)

The synthesis was performed as the synthesis of 4,4-diethoxy[1-¹³C]butane-2-one (11) using 4,4-diethoxy-1-(phenylsulfonyl) [U—¹³C₄]butane-2-one as the starting material. A solution of 4,4-diethoxy-1-(phenylsulfinyl)[1-¹³C]butan-2-one (0.5 g 1.75 mmol) and ethanol (5 mL absolute) was stirred at room temperature under a constant flow of nitrogen and a scoop of wet Raney nickel 2800 was added. The reaction mixture immediately changed from a yellow to an orange color. TLC analysis (80% EtOAc/20% Hexane) at that point showed the presence of some starting material. After about 30 minutes of reaction time, another scoop of Raney nickel was added. The reaction mixture changed from an orange to milky appearance and TLC analysis showed the complete disappearance of starting material. The Raney nickel was filtered using a frit funnel packed with celite, and the celite cake was rinsed continuously with ethanol. The filtrate was partitioned between dichloromethane (20 mL) and DI water (10 mL) and the organic layer was separated, dried over anhydrous sodium sulfate, filtered into a round bottom, and then concentrated under reduced pressure to afford 0.25 g, 89.2% of a pale yellow fluid. (Rf=0.48, 80% Hex/20% EtOAc).

The spectra data are as follows:

¹HNMR (300 MHz in CDCl₃ with 0.03% TMS) δ: 4.92, 4.90, 4.88 (t J 5.53 1H), δ: 3.69, 3.68, 3.67, 3.66, 3.65, 3.64, 3.61, 3.58, 3.56, 3.55, 3.53, 3.51, 3.50, (m pseudo chirality effect, 4H), δ: 2.76, 2.74 (d J 5.88 2H), δ: 2.39, 1.97 (d, J 127.21 ¹³CH₃), δ: 1.22, 1.19, 1.17 (t, J 6.98, 3H).

¹³CNMR (75 MHz in CDCl₃) δ: 205.96, 205.43 (d J 40.33 C═O), δ: 99.73, (CH(OR)₂) δ: 62.14, δ: 48.30, 48.13 (d J 13.08 CH₂) 31.06 (s ¹³CH₃), and 15.12.

### Synthesis of 4,4-diethoxy[U—¹³C₄]butan-2-one

The procedure above in the synthesis of 4,4-diethoxy-2[1-¹³C]-butanone was repeated with 4,4-diethoxy-1-(phenylsulfinyl)[U—¹³C]butan-2-one (210 mg, 0.729 mmol) as starring material. This experiment afforded 0.107 g, 90.23% of the title compound as a pale yellow fluid. This crude was used in subsequent reactions without further purification.

The spectra data are as follows:

¹HNMR (300 MHz in CDCl₃ with 0.03% TMS) δ: 5.19, 5.18, 5.17, 5.16, 5.15, 5.14, 4.65, 4.64, 4.63, 4.62, 4.61, 4.60, (dtd J 162.88, 5.52, 1.47 Hz, ¹³CH), δ: 3.72, −3.47 (m, pseudo chirality effect, 4H), δ: 2.98, 2.96, 2.94, 2.93, 2.56, 2.54, 2.52, 2.50 (ddd, J 127.58, 11.58, 5.88 Hz, ¹³CH₂), δ: 2.41, 2.40, 2.39, 2.38, 1.983, 1.97, 1.963, 1.960 (ddd, J 127.28, 6.28, 1.47 Hz ¹³CH₃); δ: 1.22, 1.19, 1.17 (t J 6.98 3H).

¹³CNMR (75 MHz in CDCl₃) δ: 206.25, 205.72, 205.18 (t J 40.33, ¹³C═O), 100.02, 99.41 (d 45.77 ¹³CH), δ: 62.37, 62.13, 61.87 (dd, 17.44, 19.62 OCH₂), δ: 48.85, 48.67, 48.33, 48.25, 44.15, 44.07, 47.72, 47.53 (ddd J 45.77, 40.34, 12.72 ¹³CH₂), δ: 31.41, 31.23, 30.85, 30.68 (dd J 41.41, 41.42 ¹³CH₃), δ: 15.14, 15.10 (d J 3.27 CH₃).

### Synthesis of 4,4-diethoxy-1-(phenylsulfinyl)[1-¹³C]-butan-2-ol

4,4-diethoxy-1-(phenylsulfinyl)[1-¹³C]butan-2-one (0.54 g, 1.89 mmol) and anhydrous THF (5 mL) were mixed in 100 mL round bottom flask equipped with a magnetic stir bar. After about 5 mins of stirring, sodium borohydride (0.07 g, 1.89 mmol) was added as solid to the mixture at room temperature. The reaction was allowed to stir for 3 hours and ¹³CNMR at that point showed the complete formation of the desired diastereotopic peaks. The reaction mixture was quenched in saturated ammonium chloride. The mixture was then poured into a 250 mL separatory funnel containing dichloromethane (25 mL) and DI water (10 mL). The organic layer was separated, dried over anhydrous sodium sulfate, filtered, and then concentrated using a rotary evaporator to afford 0.43 g, 80% of the titled compound as yellow oil.

### Synthesis of 1-(phenylsulfinyl) [1-¹³C]pent-3-en-2one

[1-¹³C]-methylphenyl sulfoxide (1 g 7.09 mmol) and dry THF were placed in an oven dried 100 mL round bottom flask equipped with a magnetic stir bar and a rubber septum fitted to a nitrogen inlet. The mixture was flushed under a constant flow of nitrogen and set to −78° C. using dry ice and ethanol (100%) bath. Lithium diisopropylamide (5.2 mL, of 1.5 M in THF) was added slowly for a period of 5.0 minutes and after about 45 minutes of stirring, trans-ethyl crotonate (0.6 mL, 7.8 mmol) was added slowly to the reaction mixture. ¹³CNMR of an aliquot in CDCl₃ at that point indicated the formation of the product at 65 ppm and some starting material (a ratio of 85%:15% for product and starting material respectively). The reaction mixture was then poured in to a 250 mL separatory funnel containing dichloromethane (30 mL) and DI water (15 mL). The aqueous layer was extracted and poured into another 250 mL separatory funnel containing 20 mL of dichloromethane. This mixture was acidified to a pH of 2 and the organic layer was separated, dried over anhydrous sodium sulfate, filtered, and then concentrated in vacuo to afford 1.1 g, 74.3% of a yellow oily liquid (about 10% starting material).

### Synthesis of trans-4-(phenylsulfinyl) [4-¹³C]crotonic acid methyl ester

[1-¹³C]-Methyl phenyl sulfoxide (2.5 g, 0.014 mol) and anhydrous THF (20 mL) were mixed in a 100 mL round bottom equipped with a magnetic stir bar and a rubber septum fitted to a nitrogen inlet. This mixture was stirred under a constant flow of nitrogen for a period of 10 minutes, after which it was then equilibrated at −78° C. in an ethanol dry ice bath. After about 10 minutes of equilibration, lithium diisopropylamide (17.7 mL, 1.8 eq) was added slowly for a period of 2 minutes. The resultant mixture was stirred for a period of 45 minutes to ensure complete anion formation. At that point, 3-methoxy acrylic acid methyl ester (2.09 mL, 0.015 mol) was added neat to the reaction mixture still at −78° C. Initial ¹³CNMR in CDCl₃ showed the formation of a new peak at 59 ppm and some starting material at 44 ppm (a ratio of 80% to 20% product starting material). The reaction mixture was allowed to go for an additional 3.0 hours and ¹³CNMR analysis of an aliquot in CDCl₃ at that point showed 85% conversion of starting material to product. After about 30 minutes of stirring, the reaction mixture was poured into a separatory funnel containing dichloromethane (75 mL) and deionised water (30 mL). This mixture was acidified to a pH of 2 and the organic layer was extracted (2×50 mL). The combined organic layers were dried over anhydrous sodium sulfate, filtered, and then concentrated using a rotary evaporator to afford 4.2 g of a red fluid. This crude product was purified by dry column chromatography (using 80% EtoAc/20% hexane as the eluent) to afford 2.74 g, 86.9% of the titled compound light red oil, which immediately solidified on standing.

The spectra data are as follows:

¹HNMR δ: 7.58-7.52 (m 5H); δ: 6.73-6.65 (m unresolved multiplet 1H); δ: 5.93-5.86 (ddd J 15.44, 6.98, 6.25, 1.1); δ: 3.99-3.31 (two sets of ddd J 149.28, 12.87, 7.72, 2H); δ: 3.72 (s 3H).

¹³CNMR (75 MHz in CDCl₃) δ: 138.60, 131.66, 129.43, 128.48, 59.04 and 55.13 (the carbonyl peak was not seen).

### Synthesis of trans-4-ethoxy-1-(phenylsulfinyl)-3[1-¹³C]buten-2-one

A mixture of 4,4 diethoxy-1-(phenyl sulfinyl)[1-¹³C]-butan-2-one (110 mg, 0.385 mmol), sodium acetate (0.031 g catalytic amount) and toluene (2 mL) was heated under reflux and stirring for 14 hrs. ¹³CNMR analysis after this period confirmed 80% conversion of starting material to product. The reaction was allowed to go for an additional 4 hours. After this period, there was no noticeable change in the extent of the reaction. The heating was discontinued and the mixture was allowed to reach room temperature. After cooling to room temperature, the entire mixture was partitioned between dichloromethane (10 mL) and DI water (10 mL). The organic layer was separated, dried over anhydrous sodium sulfate, filtered, and concentrated under reduced pressure to afford 100 mg of crude product. The crude product was chromatographed (using silica, and 100% ethyl acetate) to afford 56 mg 62% of pure product.

The spectra data are as follows:

¹HNMR (300 MHz in CDCl₃ with 0.03% TMS) δ: 7.689, 7.679, 7.6750, 7.665, 7.657, 7.534, 7.5241, 7.528, 7.518, 7.510 (5H, m), δ: 7.599, 7.557 (1H, d J 12.50 typical of trans). δ: 5.667, 5.662, 5.625, 5.621 (1H dd J 12.51, 1.48); δ: 4.22, 4.176, 3.754, 3.709 (¹³CH dd J 140.08, 13.23) δ: 4.04, 3.99, 3.57, 3.532 (¹³CH dd J 140.08, 13.23). δ: 3.992, 3.968, 3.943, 3.920 (OCH₂ q J 7.35); δ: 1.368, 1.343, 1.320 (CH₃ t J 6.99).

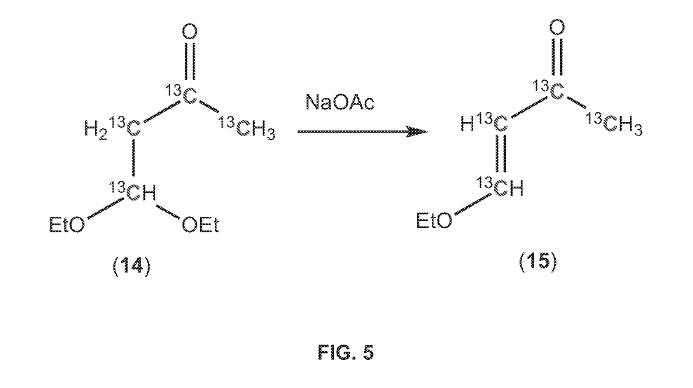

FIG. 5 illustrates obtaining 4-ethoxy-3-[U-13C4]butene-2-one (15) in accordance with the disclosed embodiments. The 4,4-diethoxy[U-13C4]butane-2-one (14) can be treated with sodium acetate to yield 4-ethoxy-3-[U-13C4]butene-2-one (15) as shown in FIG. 5, as follows:

### General procedure for the synthesis of trans-4-alkoxy-3-[1-¹³C]butene-2-one (12, 15)

4,4-diethoxy[1-¹³C]butan-2-one and sodium acetate in toluene were heated under reflux and stirred for 14 hours. The product is produced in quantitative yield.

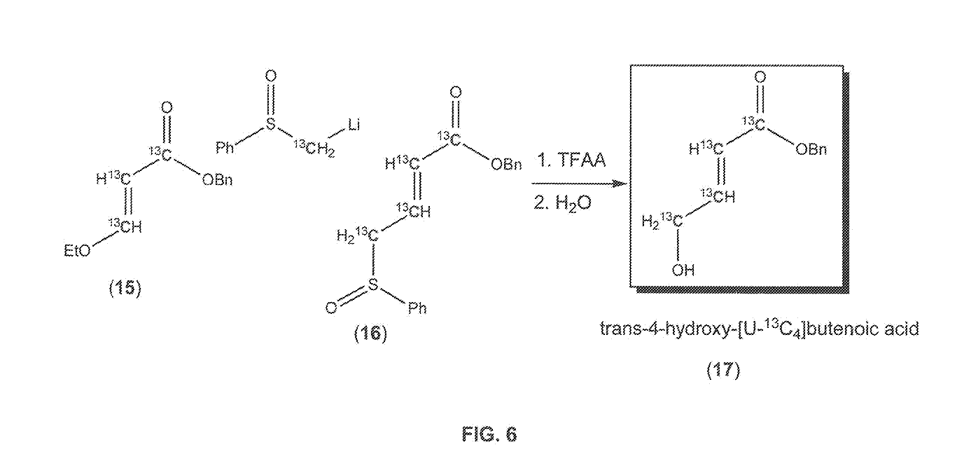

FIG. 6 illustrates obtaining trans-4-hydroxy-2-buteneoic acid in accordance with the disclosed embodiments. Trans-4-hydroxy-2-buteneoic acid can be produced by two procedures. If the trans-4-hydroxy-2-buteneoic acid (17) is required, the pathway shown in FIG. 5 is preferred. The product produced from this reaction is only the trans-4-hydroxy-2-buteneoic acid (17).

### Synthesis of 4-Phenylsulfinyl-2[1,3,4-¹³C₃]butenoic acid (16)

Ethoxy phenylsulfonyl[¹³C]methane (5.2 g, 25.9 mmol, 1.0 equivalent) was weighed out in a five-hundred milliliter Morton flask, then flushed with argon. It was then dissolved in tetrahydrofuran (50 mL) before the flask was cooled using a dry-ice/200 proof ethanol bath. After ten minutes of stirring, lithium diisopropylamide (1.5M soln. in THF/cyclohexane, 37.94 mL, 56.9 mmol, 2.2 eq) was added slowly while stirring vigorously. The reaction mixture was kept at −78° C. for an hour to ensure anion formation. In the meantime, bromo[1-¹³C]acetic acid (3.98 g, 28.5 mmol, 1.1 eq) was weighed out in a one-hundred milliliter Morton flask, then flushed with argon. It was then dissolved in tetrahydrofuran (10 mL) and stirred vigorously. After the hour was up, the bromo[1-¹³C]acetic acid solution was slowly added to the main reaction mixture. After one more hour of stirring at −78° C., more LDA (18.97 mL, 28.5 mmol, 1.1 eq) was added. The reaction was monitored by ¹³CNMR by taking an aliquot from the reaction mixture, quenching with 1N HCl, extracting with CH₂Cl₂, and drying sample with sodium sulfate before concentrating on rotovap. The disappearance of (to within <2% remaining) ethoxy phenylsulfonyl[¹³C]methane (δ=86 ppm) and subsequent appearance of 3-ethoxy-2-[1, 3-¹³C₂]propenoic acid (δ=158, 175 ppm) was monitored. In the meantime, [¹³C]methyl phenyl sulfoxide (3.65 g, 25.9 mmol, 1.0 eq) was weighed out in a one-hundred milliliter Morton flask, then flushed with argon. It was then dissolved in tetrahydrofuran (30 mL) and stirred vigorously prior to submerging the flask in a dry-ice/200 proof ethanol bath. After ten minutes of stirring, lithium diisopropylamide (1.5M soln. in THF/cyclohexane, 18.97 mL, 28.5 mmol, 1.1 eq) was added slowly while stirring vigorously. After one hour since the last LDA addition to the 3-ethoxy-2-[1,3-¹³C₂]propenoic carboxylate reaction, the [¹³C]methyl phenyl sulfoxide anion was slowly added to carboxylate reaction mixture. The reaction was allowed to reach room temperature as the dry ice evaporated. After 20 hours, the reaction was found not yet to be complete by ¹³CNMR by taking an aliquot from the reaction mixture, quenching with 1N HCl, extracting with CH₂Cl₂, and drying sample with sodium sulfate before concentrating on rotovap. The disappearance of 3-ethoxy-2-[1,3-¹³C₂]propenoic carboxylate (δ=158, 175 ppm) and subsequent appearance of the desired 4-phenylsulfinyl-2-[1,3,4-¹³C₃]butenoic acid (δ=59, 136, 169 ppm) was monitored. At this point there was only a 22% conversion to product, so reaction was heated at 57 to 70 degrees Celsius for a total of 22 hours to achieve a 70% conversion to product. The reaction mixture was worked up in the same manner as aliquot, using 3×60 mL of CH₂Cl₂ to extract. Volatiles were then removed by vacuum using a rotary evaporator to give a golden-brown viscous oil (2.09 g, 38%). Crude product was used without further purification.

The spectra data are as follows:

¹HNMR (CDCl₃, 300 MHz): δ=3.3-4.3 (m, 2H), 5.7-6.0 (m, 1H), 6.3-6.6 (m, 0.5H), 6.8-7.1 (m, 0.5H), 7.2-8.0 (m, Ar), 10.4-11.0 (br, OH).

¹³CNMR (CDCl₃, 75 MHz): δ=58.17, 58.27, 58.75, 58.85 (m, J=44 Hz, ¹³CH₂), 63 (m, CH), 135.5, 136.1 (d, J=44 Hz, ¹³CH), 168.82, 168.91 (d, J=6.7 Hz, ¹³C═O).

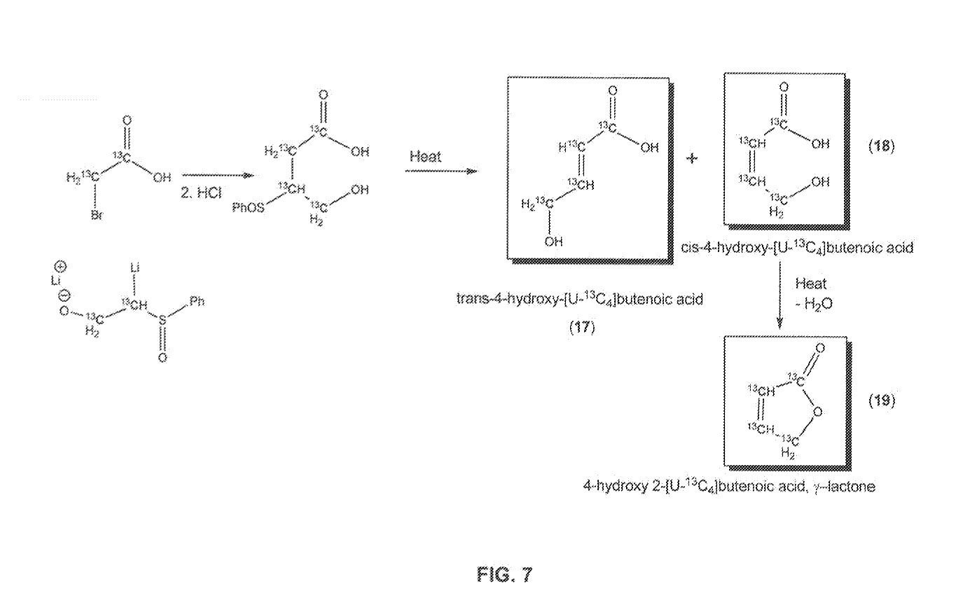

FIG. 7 illustrates obtaining cis and trans 4-hydroxy-2-butenoic acid in accordance with the disclosed embodiments. The second pathway for production of trans 4-hydroxy-2-butenoic acid (17) provides for the synthesis of both cis and trans 4-hydroxy-2-butenoic acid which allows for the facile separation of the cis and trans isomers by esterification of the cis compound, as follows:

### Synthesis of Methyl 4-(Phenylsulfinyl) 2-[3,4-¹³C₂]butenoate (17)

[¹³C]Methyl phenyl sulfoxide (1.07 g, 7.6 mmol, 1.0 equivalent) was weighed out in a one-hundred milliliter Morton flask, then flushed with argon. It was then dissolved in tetrahydrofuran (10 mL) before the flask was cooled using a dry-ice/200 proof ethanol bath. After ten minutes of stirring, lithium diisopropylamide (1.5M soln. in THF/cyclohexane, 7.63 mL, 11.4 mmol, 1.5 eq) was added slowly while stirring vigorously. The reaction mixture progressively turned thick orange and was kept at −78° C. for an hour to ensure anion formation. In the meantime, 3-ethoxy-methyl-2-[3-¹³C]propenoate (1.0 g, 7.6 mmol, 1.0 eq) was weighed out in a one-hundred milliliter Morton flask, then flushed with argon. It was then dissolved in tetrahydrofuran (5 mL) and stirred vigorously. After the hour was up, the propenoate was slowly added to the main reaction mixture, progressively turning the reaction a red-brown color. The reaction was allowed to reach room temperature as the dry ice evaporated. After 18 hours, the reaction was found to be complete by ¹³CNMR by taking an aliquot from the reaction mixture, quenching with 1N HCl, extracting with CH₂Cl₂, and drying sample with sodium sulfate before concentrating on rotovap. The disappearance of (to within <2% remaining) 3-ethoxy-methyl-2-[3-¹³C]propenoate (δ=162 ppm) and subsequent appearance of the desired 4-phenylsulfinyl-methyl-2-[3,4-¹³C₂]butenoate (δ=60, 134 ppm) was monitored. The reaction mixture was worked up in the same manner, using 3×30 mL of CH₂Cl₂ to extract. Volatiles were then removed by vacuum using a rotary evaporator to give an amber oil (943 mg, 54%). Crude product was used without further purification.

The spectra data are as follows:

¹HNMR (CDCl₃, 300 MHz): δ=3.5-4.3 (m, 2H), 3.7 (s, 3H), 5.7-6.0 (m, 1H), 6.3-6.5 (m, 0.5H), 6.8-7.1 (m, 0.5H), 7.4-7.8 (m, Ar).

¹³CNMR (CDCl₃, 75 MHz): δ=59.5 (d, ¹³CH₂), 63 (CH), 134.2 (d, ¹³CH), 160 (C═O).

### Synthesis of 4-hydroxy-2-[¹³C₄]butenoic acid (18)

Methyl 4-(Phenylsulfinyl) 2-[3,4-¹³C₂]butenoate (100 mg, 0.44 mmole) was dissolved in CDCl₃ (2.5 mL) under nitrogen. The reaction was cooled, stirred in an ice-water bath while purging with nitrogen. Then trans-4-hydroxy-2-buteneoic acid was treated with TFAA (2.5 mL). Five minutes later, the reaction was found to be complete. The TFAA evaporated, the resulting oil was dissolved in ethanol/water, and sodium borohydride was added. The reaction was complete within five minutes. The reaction mixture was acidified to a pH=2, then extracted into dichloromethane. The organic layer was dried over anhydrous sodium sulfate, and removed by vacuum to give the title compound in quantitative yield.

The spectra data are as follows:

¹HNMR (CDCl₃, 300 MHz): δ=3.5-4.3 (m, 2H), 3.7 (s, 3H), 5.7-6.0 (m, 1H), 6.3-6.5 (m, 0.5H), 6.8-7.1 (m, 0.5H), 7.4-7.8 (m, Ar).

¹³CNMR (CDCl₃, 75 MHz): δ=65.5 (d, ¹³CH₂), 63 (CH), 134.2 (d, ¹³CH), 160 (C═O).

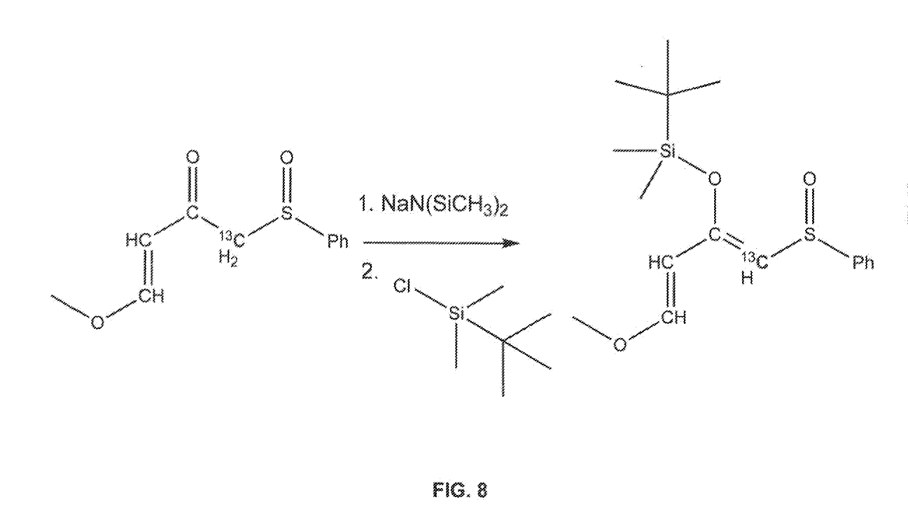

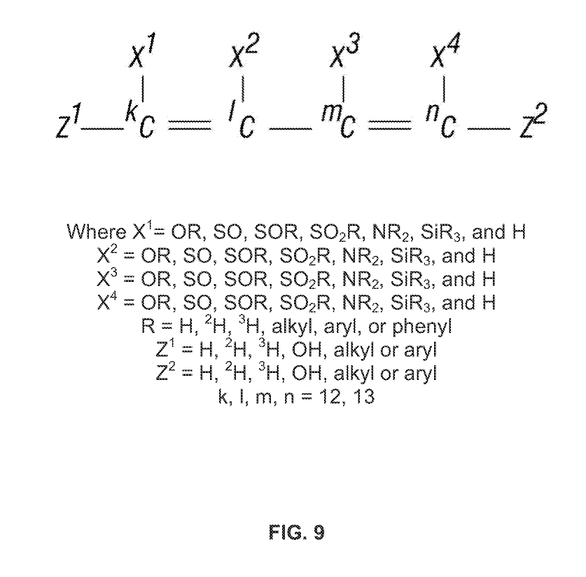

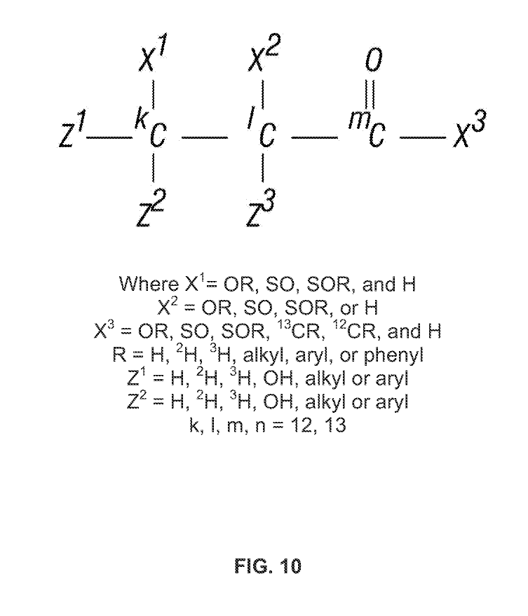

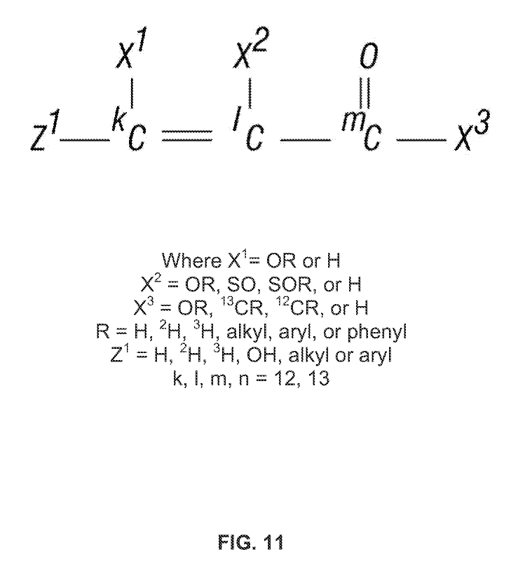

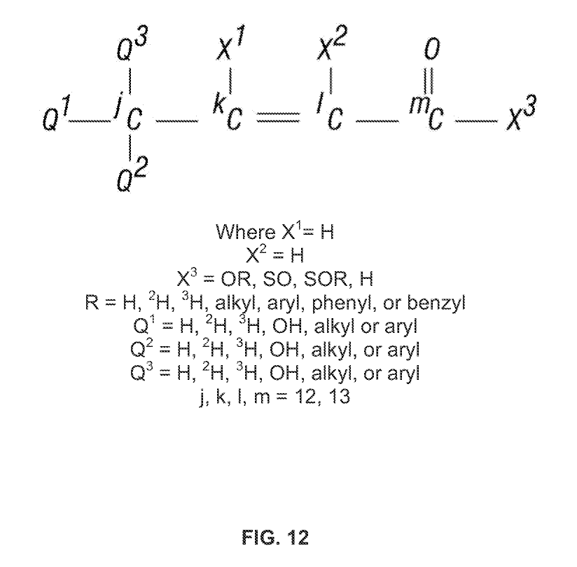

FIG. 8 illustrates obtaining (E,Z) 1-methoxy-3-t-butyldimethylsiloxy-4-(phenylsulfinyl)[4-¹³C]1,3-butadiene in accordance with the disclosed embodiments as follows:

### Synthesis of (E,Z) 1-methoxy-3-t-butyldimethylsiloxy-4-(phenylsulfinyl[4-¹³C]1,3-butadiene

A solution of sodium hexamethyldisilazide in toluene (0.6 M 8.2 mL, 4.88 mmol) was diluted with THF (12 mL) and cooled to −78° C. Added to the resulting solution was the trans-4-methoxy-1-(phenylsulfinyl)-3-[1-¹³C]buten-2-one (0.9 g, 4.66 mmol) in THF (5 mL) over a period of 5 minutes. The reaction was warmed to −30° C. over a period of 1.5 hours then cooled back to −78° C. The resultant reaction mixture was treated with t-butyldimethylsilyl chloride (0.77 g, 5.12 mmol) in 5 mL THF. The mixture was again warmed up to room temperature, diluted with 50 mL of ether, and filtered through a frit funnel packed with dry celite. The resultant filtrate was concentrated in vacuo to afford 1.53 g of the titled compound as a mixture with hexamethyldisilazine and toluene. This mixture was subjected to high vacuum treated for 24 hours which resulted in 1.50 g of the titled compound still as a mixture.

The spectra data are as follows:

¹HNMR (300 MHz in CDCl₃ with 0.03% TMS) δ: 7.61-7.22 (m, 5H), δ: 7.05, 7.00 (d, J 12.87 Hz, 1H); δ:5.80, 5.23 (d J 175 Hz, 1H), δ:5.29, 5.28, 5.24, 5.23 (dd J 12.5 2.94 Hz 1H); δ: 3.61 (s 3H); δ:1.05, 0.91 (9H); δ:0.37 0.28 (6H).

¹³CNMR δ: 155.80, 155.10, 146.26, 145.91 129.91, 129.00, 128.94, 12819, 133.46; 111.60, 101.72, 101.59, 56.95, −1.49, −3.58, −3.7, −4.45.

What is claimed is:

1. A labeled compound having the structure: 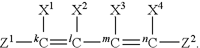
wherein k=12 or 13, l=12 or 13, m=12 or 13, and n=12 or 13, with the proviso that k, l, m, and n do not simultaneously equal 12; wherein X¹ is selected from the group consisting of OR, SO, SOR, SO₂R, NR₂, SiR₃, and H; wherein X² is selected from the group consisting of OR, SO, SOR, SO₂R, NR₂, SiR₃, and H; wherein X³ is selected from the group consisting of OR, SO, SOR, SO₂R, NR₂, SiR₃, and H; wherein X⁴ is selected from the group consisting of OR, SO, SOR, SO₂R, NR₂, SiR₃, and H; wherein R is selected from the group consisting of H, ²H, ³H, alkyl, aryl, or phenyl; wherein Z¹ is selected from the group consisting of H, ²H, ³H, alkyl or aryl; and, wherein Z² is selected from the group consisting of H, ²H, ³H, alkyl or aryl.

2. The labeled compound of claim 1 wherein k=12, l=12, m=12, n=13, X¹═O-alkyl, X²═H, X³=t-butyldimethylsiloxy, X⁴═SO-phenyl, Z¹═H, and Z²═H.

3. A labeled compound having the structure: 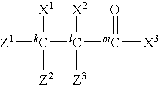
wherein k=12 or 13, l=12 or 13, m=12 or 13, and n=12 or 13, with the proviso that k, l, m, and n do not simultaneously equal 12; wherein X¹ is selected from the group consisting of OR, SO, SOR, and H; wherein X² is selected from the group consisting of OR, SO, SOR, and H; wherein X³ is selected from the group consisting of OR, SO, SOR, ¹³CR, ¹²CR, and H; wherein R is selected from the group consisting of H, ²H, ³H, alkyl, aryl, or phenyl; wherein Z¹ is selected from the group consisting of H, ²H, ³H, alkyl or aryl; and wherein Z² is selected from the group consisting of H, ²H, ³H, alkyl or aryl.

4. The labeled compound of claim 3 wherein k=13, l=13, m=13, X¹═O-alkyl, X²═O-alkyl, X³═O-alkyl, Z¹═H, Z²═H, and Z³═H.

5. The labeled compound of claim 3 wherein k=12, l=12, m=12, X¹═O-alkyl, X²═O-alkyl, X³=ⁿCH₂—SO-phenyl, wherein n=13, Z¹═H, Z²═H, and Z³═H.

6. The labeled compound of claim 3 wherein k=12, l=12, m=12, X¹═O-alkyl, X²═O-alkyl, X³=ⁿCH₃, wherein n=13, Z¹═H, Z²═H, and Z³═H.

7. The labeled compound of claim 3 wherein k=13, l=13, m=13, X¹═O-alkyl, X²═O-alkyl, X³=ⁿCH₂—SO-phenyl, wherein n=13, Z¹═H, Z²═H, and Z³═H.

8. The labeled compound of claim 3 wherein k=13, l=13, m=13, X¹═O-alkyl, X²═O-alkyl, X³=ⁿCH₃, wherein n=13, Z¹═H, Z²═H, and Z³═H.

9. A labeled compound having the structure: 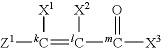
wherein k=12 or 13, l=12 or 13, and m=12 or 13, with the proviso that k, l, and m, do not simultaneously equal 12; wherein X¹ is selected from the group consisting of OR and H; wherein X² is selected from the group consisting of OR, SO, SOR, and H; wherein X³ is selected from the group consisting of OR, ¹³CR, ¹²CR, and H; wherein R is selected from the group consisting of H, ²H, ³H, alkyl, aryl, and phenyl; and wherein Z¹ is selected from the group consisting of H, ²H, ³H, alkyl or aryl.

10. The labeled compound of claim 9 wherein k=13, l=13, m=13, X¹═O-alkyl, X²═O-alkyl, and Z¹═H.

11. The labeled compound of claim 9 wherein k=13, l=13, m=13, X¹═O-alkyl, X²=ⁿCH₃, wherein n=13, and Z¹═H.

12. The labeled compound of claim 9 wherein k=13, l=13, m=13, X¹═O-alkyl, X²=ⁿCH₃, wherein n=13, and Z¹═H.

13. A labeled compound having the structure: 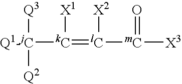
wherein j=12 or 13, k=12 or 13, l=12 or 13, and m=12 or 13, with the proviso that j, k, l, and m do not simultaneously equal 12; wherein X¹═H; wherein X²═H; wherein X³ is selected from the group consisting of OR, SO, SOR, and H; wherein R is selected from the group consisting of H, ²H, ³H, alkyl, aryl, phenyl, and benzyl; wherein Q¹ is selected from the group consisting of H, ²H, ³H, OH, alkyl or aryl; wherein Q² is selected from the group consisting of H, ²H, ³H, OH, alkyl or aryl; and wherein Q³ is selected from the group consisting of H, ²H, ³H, OH, alkyl or aryl.

14. The labeled compound of claim 13 wherein j=13, k=13, l=13, m=13, X¹═H, X²═H, X³═O-benzyl, Q¹═H, Q²═OH, and Q³═H.

15. The labeled compound of claim 13 wherein j=13, k=13, l=13, m=13, X¹═H, X²═H, X³═OH, Q¹═H, Q²═OH, and Q³═H.
